SEQUENCE LISTING (2 sequences)

SEQ ID NO: 1 - protein, 29 aa, Homo sapiens
HSQGTFTSDYSKYLDSRRAQDFVQWLMNT

SEQ ID NO: 2 - protein, 180 aa, Homo sapiens
MKSIYFVAGLFVMLVQGSWQRSLQDTEEKSRSFSASQADPLSDPDQMNEDKRHSQGTFTSDYSKYLDSRRAQDFVQWLMNTKRNRNNIAKRHDEFERHAEGTFTSDVSSYLEGQAAKEFIAWLVKGRGRRDFPEEVAIVEELGRRHADGSFSDEMNTILDNLAARDFINWLIQTKITDRK

(19) United States
(12) Patent Application Publication
(10) Pub. No.: US 2015/0216940 A1
(43) Pub. Date: Aug. 6, 2015

(54) NANOPARTICLE GLUCAGON COMPOSITIONS

(71) Applicant: Midatech Limited, Abingdon (GB)

(72) Inventor: Thomas Rademacher, Oxfordshire (GB)

(21) Appl. No.: 14/608,311

(22) Filed: Jan. 29, 2015

(30) Foreign Application Priority Data
Jan. 31, 2014 (GB) 1401708.1

(51) Int. Cl. A61K 38/26; A61K 9/10; A61K 47/48

(52) U.S. Cl. CPC A61K 38/26; A61K 47/48884; A61K 9/10; A61K 47/48092; A61K 47/48038

(57) ABSTRACT

The present invention relates to glucagon peptide-carrying nanoparticles, particularly for use in medicine, and includes methods for treatment of hypoglycaemia, such as a diabetic hypoglycaemic adverse event. Nanoparticle composition comprise a nanoparticle comprising a core comprising a metal and/or a semiconductor; and a corona comprising a plurality of ligands covalently linked to the core, wherein said plurality of ligands comprise at least one glutathione; and at least one glucagon peptide that is non-covalently bound to the corona.

### FIELD OF THE INVENTION

The present invention relates to peptide-carrying nanoparticles, particularly for use in medicine, and includes methods for treatment of disorders, e.g., of hypoglycaemia.

### BACKGROUND TO THE INVENTION

The present invention is directed at compositions and products, and methods of making and administering such compositions and products, including for the treatment of mammals and particularly humans.

Bioactive agents, such as peptides, frequently suffer from poor stability, particularly thermo-stability, which may limit the conditions to which the agents can be subjected during preparation, processing, storage and/or delivery. Medical preparations of peptides for human use are generally formulated with one or more preservatives and/or stabilisers. Moreover, limited gastrointestinal stability typically presents a barrier to effective oral administration of bioactive peptides.

WO 2011/154711 describes glyconanoparticles that have a gold core surrounded by a carbohydrate corona and which act as carriers for peptides such as insulin.

Glucagon, a 29 amino acid peptide derived from proglucagon, is a life-saving medication used in the treatment of hypoglycaemia. In particular, hypoglycaemia such as diabetic hypoglycaemia, can render the subject unconscious or otherwise unable to take glucose orally. Glucagon is typically administered by intramuscular, intravenous or subcutaneous injection. Glucagon quickly elevates blood glucose levels by promoting gluconeogenesis and glycogenolysis, whereby glucose is liberated from liver glycogen stores.

A particular problem with the medical use of glucagon is that it suffers from poor solubility in aqueous buffers at or near physiological pH. Moreover, glucagon exhibits poor stability in solution, forming multiple degradation-related peptides and/or amyloid fibrils. Consequently, medicinal preparations of glucagon are typically in the form of a lyophilized solid to be reconstituted as a solution immediately prior to use. Unused solution therefore must be discarded. The reconstitution process is cumbersome, particularly for a hypoglycaemic patient, which can make self-administration difficult or impossible.

Moreover, certain formulations of glucagon require a “cold chain”; a requirement that is difficult or impossible to achieve in some medical settings, for example in the third world or in-the-field settings.

There remains an unmet need for further nanoparticle compositions capable of carrying bioactive peptides, and for methods of delivering such bioactive peptides to a subject.

The present invention addresses these and other needs.

### BRIEF DESCRIPTION OF THE INVENTION

Broadly, the present invention relates to glucagon-carrying nanoparticle compositions. The present inventors have found that nanoparticles having a mixed corona of glutathione ligands and glucose-containing ligands bind glucagon (in some cases with a binding capacity of around 30 glucagon molecules per nanoparticle). Nanoparticles as defined herein therefor provide a carrier for the formulation and delivery of glucagon to subjects in need of glucagon treatment, e.g. for rapid treatment of hypoglycaemia.

The stabilising and solubilising effect of the nanoparticle carriers of glucagon peptide facilitates administration, e.g., by avoiding a preparatory step of reconstituting lyophilised glucagon that is necessary with conventional glucagon formulations. This in turn improves storage and facilitates faster administration of glucagon, which can be time-critical, for example when a subject is experiencing a fast onset of hypoglycaemia and risks unconsciousness, coma and even death.

Accordingly, in a first aspect the present invention provides a nanoparticle composition comprising:
(a) a nanoparticle comprising:
   (i) a core comprising a metal and/or a semiconductor;
   (ii) a corona comprising a plurality of ligands covalently linked to the core, wherein said plurality of ligands comprise at least one glutathione; and
(b) at least one glucagon peptide that is non-covalently bound to the corona.

In accordance with any one of the aspects of the present invention, the corona may comprise one or more carbohydrate ligands, for example one or more monosaccharide ligands, covalently attached to the core via a linker.

In accordance with any one of the aspects of the present invention, the glucagon peptide may comprise or consist of an amino acid sequence having at least 70%, 80%, 90%, 95% or 99% amino acid sequence identity with the full-length amino acid sequence set forth as SEQ ID NO: 1. In some cases, the glucagon peptide has up to 1, 2, 3, 4 or 5 amino acid changes by substitution, addition and/or deletion as compared with the full-length amino acid sequence set forth in SEQ ID NO: 1. In some cases, the glucagon peptide comprises or consists of the full-length amino acid sequence HSQGTFTSDYSKYLDSRRAQDFVQWLMNT (SEQ ID NO: 1).

SEQ ID NO: 1 is the 29 amino acid sequence of residues 53-81 of the complete 180 amino acid sequence of the human glucagon gene product set forth below as SEQ ID NO: 2 and disclosed under UniProt accession no. P01275, version 3, dated 6 Feb. 2007 (checksum: 7A99EEC629B2862C).

>sp|P01275|GLUC_HUMAN Glucagon OS=Homo sapiens GN=GCG PE=1 SV=3

(SEQ ID NO: 2)
MKSIYFVAGLEVMLVQGSWQRSLQDTEEKSRSFSASQADPLSDPDQMNED
KRHSQGTFTSDYSKYLDSRRAQDFVQWLMNTKRNRNNIAKRHDEFERHAE
GTFTSDVSSYLEGQAAKEFIAWLVKGRGRRDFPEEVAIVEELGRRHADGS
FSDEMNTILDNLAARDFINWLIQTKITDRK.

The 29 amino acid sequence of the human glucagon peptide (residues 53-81) is shown underlined (SEQ ID NO: 1).

In certain cases in accordance with the present invention, the glucagon peptide may be selected from the group consisting of:
(i) a peptide comprising or consisting of an amino acid sequence having at least 70%, 80%, 90%, 95% or 99% amino acid sequence identity to the full-length sequence set forth in SEQ ID NO: 1;
(ii) a peptide comprising or consisting of the full-length amino acid sequence set forth in SEQ ID NO: 1;
(iii) a peptide comprising or consisting of a variant sequence of the full-length amino acid sequence set forth in SEQ ID NO: 1, wherein said variant differs by addition, deletion, substitution or modification (e.g. chemical modification or derivatisation) of not more than 1, 2, 3, 4, 5, 6, 7, 8, 9 or not more than 10 amino acids from said full-length amino acid sequence set forth in SEQ ID NO: 1;
(iv) a peptide comprising or consisting of a contiguous fragment of any one of (i)-(iii), said fragment having a sequence length of at least 15, 20, 25 or 28 amino acids.

Preferably, the glucagon peptide exhibits biological activity of glucagon. In particular, said glucagon peptide of any one of (i)-(iv) may exhibit at least 50% of the activity of the glucagon peptide of SEQ ID NO: 1 in an in vitro or in vivo bioassay of glucagon activity. In certain cases, the glucagon activity may comprise one or more activities selected from the group consisting of: glucagon receptor agonist activity (e.g. activation of the human glucagon receptor having the amino acid sequence set forth at UniProt accession no. P47871, version 1, 1 Feb. 1996); stimulation of gluconeogenesis; stimulation of glycogenolysis; elevation of blood glucose concentration; and suppression of liver glycolysis. In particular, the glucagon peptide activity may comprise the ability to restore normal blood glucose concentration in a hypoglycaemic mammal, e.g., a diabetic human experiencing a hypoglycaemic episode.

It has been found that the nanoparticles in accordance with the present invention may be provided with a variety of numbers of ligands forming the corona. For example, in some cases the corona comprises at least 5, 10, 20 or at least 50 ligands per core, e.g. between about 10 to about 1000 ligands per core. In particular, the nanoparticle compositions in accordance with any aspect of the present invention may comprise at least 5, 10, 15, 20 or at least 50 glutathione ligands and/or carbohydrate ligands per core.

The number of glucagon peptide molecules bound per core is not particularly limited. For certain applications, it may be desirable to employ as few as 1, 2, 3 or 4 glucagon peptides per core, while in other cases the nanoparticle of the invention may comprise at least 5, 10, 15, 20, 30 or at least 50 or more glucagon peptide molecules bound per core. In particular cases, the mean number of glucagon peptides per core is approximately 30, e.g., within the range 25-35.

In accordance with any one of the aspects of the present invention, the glucagon peptide may in some cases be monomeric.

In some cases, in accordance with any one of the aspects of the present invention, the at least one glucagon peptide may be bound to the corona of the nanoparticle in a reversible manner. In particular, the glucagon peptide may be bound to the corona such that at least a fraction of the bound glucagon peptide is released from the nanoparticle upon contacting the nanoparticle with a physiological solution. The glucagon peptide may in some cases be adsorbed to the corona of the nanoparticle. The glucagon peptide may in some cases be electrostatically or otherwise non-covalently bound to the one or more ligands that form the corona of the nanoparticle.

In some cases, in accordance with any one of the aspects of the present invention, said ligands comprise glutathione alone or in conjunction with other species of ligand, e.g., combinations of glutathione and carbohydrate ligands (including glucose-containing ligands) are specifically contemplated herein.

In some cases, in accordance with any one of the aspects of the present invention, the nanoparticle comprises at least 10, at least 20, at least 30, at least 40 or at least 50 ligands which are (i) glutathione ligands; or (ii) both glutathione ligands and ligands other than glutathione, such as carbohydrate-containing ligands.

In some cases in accordance with any one of the aspects of the present invention, the corona comprises at least two different species of ligands in a specific molar ratio. For example, the corona may comprise glutathione ligands as a first species and carbohydrate (e.g. glucose)-containing ligands as a second species, wherein the first and second species are present in a ratio of between 100:1 and 1:1, such as between 50:1 and 5:1 or between 20:1 and 8:1. In certain cases, the corona of the nanoparticles comprises 90% glutathione ligands and 10% glucose-containing ligands (e.g. glucose covalently attached to the core via a C2 thiol linker).

In some cases, in accordance with any one of the aspects of the present invention, the diameter of the core of the nanoparticle is in the range 1 nm to 5 nm.

In some cases, in accordance with any one of the aspects of the present invention, the diameter of the nanoparticle including its ligands is in the range 2 nm to 50 nm, optionally 3 nm to 30 nm, or 4 nm to 20 nm, or 5 nm to 15 nm.

In some cases, in accordance with any one of the aspects of the present invention, the core comprises a metal selected from the group consisting of: Au, Ag, Cu, Pt, Pd, Fe, Co, Gd and Zn, or any combination thereof.

In some cases, in accordance with any one of the aspects of the present invention, the core is magnetic.

In some cases, in accordance with any one of the aspects of the present invention, the core comprises a semiconductor. The semiconductor may comprise metal atoms, such as cadmium. Alternatively or additionally, the semiconductor may comprise non-metal atoms. Organic semiconductors are specifically contemplated herein. Preferred semiconductors, in accordance with the present invention, may be selected from the group consisting of: cadmium selenide, cadmium sulphide, cadmium tellurium and zinc sulphide.

In some cases, in accordance with any one of the aspects of the present invention, the core is capable of acting as a quantum dot.

Preferably, the composition in accordance with the first aspect of the invention comprises a plurality, e.g., 100, 1000, 100000, or more, of said nanoparticles, wherein at least 10%, 20%, 30%, 40%, 50%, 60%, 70%, 80%, 90% or 95% of the nanoparticles in said composition have at least one glucagon peptide bound.

In some cases, in accordance with any one of the aspects of the present invention, the nanoparticle composition comprises a carrier, such as solution, a polymer (e.g. a viscoelastic polymer film), a powder, or a cream, in which the nanoparticles and bound glucagon peptides are suspended and/or embedded. In certain cases, the composition may be in the form of a patch or film for delivery to or across skin, mouth, cheek (transbuccal), vagina, rectum or in the form of a spray for delivery into the mouth, nose, lungs or the rectum or vagina. The composition may be in an associated form, a suspension or contained together in a single package, container or carrier. In certain cases, the composition may take the form of one or more doses (e.g. a defined quantity of glucagon peptide or glucagon peptide activity units), such as in the form of a therapeutic dose or defined number of doses. In certain cases the composition may be in the form of a viscoelastic polymer film for transbuccal delivery.

In some cases, in accordance with any one of the aspects of the present invention, the nanoparticle composition further comprises at least one permeation enhancer that is non-covalently or covalently bound to said core and/or or said corona. As described in co-pending GB patent application No. 1301991.4, filed 5 Feb. 2013, the entire contents of which are expressly incorporated herein by reference for all purposes, certain permeation enhancers may be advantageously bound to the nanoparticle without displacing any significant active peptide, such as the glucagon peptide as defined herein. In certain cases, said permeation enhancer is selected from tetradecyl-D-maltoside and lysalbinic acid. In certain cases, said permeation enhancer, e.g. tetradecyl-D-maltoside and/or lysalbinic acid is non-covalently bound to said corona.

In a second aspect, the present invention provides a nanoparticle composition as defined in accordance with the first aspect, for use in medicine.

In a third aspect, the present invention provides a nanoparticle composition as defined in accordance with the first aspect, for use in a method of therapeutic treatment of hypoglycaemia in a mammalian subject. In some cases said hypoglycaemia may be a hypoglycaemic adverse event in a diabetic mammalian subject requiring acute treatment to elevate and/or restore blood glucose concentration.

In a fourth aspect, the present invention provides use of a nanoparticle composition as defined in accordance with the first aspect in the preparation of a medicament for therapeutic treatment of hypoglycaemia in a mammalian subject.

In a fifth aspect, the present invention provides a method of therapeutic treatment of hypoglycaemia in a mammalian subject, the method comprising administering a therapeutically effective amount of a nanoparticle composition as defined in accordance with the first aspect to the subject in need of said treatment.

In a sixth aspect, the present invention provides a method of increasing blood glucose concentration in a mammalian subject, the method comprising administering an effective amount of a nanoparticle composition as defined in accordance with the first aspect to the subject.

In accordance with any one of the second to sixth aspects of the invention, the subject may be a human, a companion animal (e.g. a dog or cat), a laboratory animal (e.g. a mouse, rat, rabbit, pig or non-human primate), a domestic or farm animal (e.g. a pig, cow, horse or sheep). Preferably, the subject is a human. In some cases the subject has type 1 diabetes, type 2 diabetes or prediabetes. In some cases in accordance with any one of the second to sixth aspects of the invention, said hypoglycaemia may be a hypoglycaemic adverse event in a diabetic mammalian subject requiring acute treatment to elevate and/or restore blood glucose concentration.

In accordance with any one of the second to sixth aspects of the invention, the subject may in certain cases have a disorder that results in abnormally lowered blood glucose concentration (i.e. hypoglycaemia). Hypoglycaemia may be a temporary state resulting from poor management of a diabetic condition, for example where too much insulin has been administered or insufficient food taken in or the insulin and food intake have been poorly timed such that the subject enters a state of hypoglycaemia.

In accordance with any one of the second to sixth aspects of the invention, it is specifically contemplated that, in some cases, the composition of the invention may be self-administered or for self-administration. The stabilising and solubilising effect of the nanoparticle carriers of glucagon peptide facilitates administration, e.g., by avoiding a preparatory step of reconstituting lyophilised glucagon that is necessary with conventional glucagon formulations. In other words, the nanoparticle glucagon composition of the invention may, in some cases, be provided in a “ready to use” form. This in turn facilitates faster administration of glucagon, which can be time-critical, for example when a subject is experiencing a fast onset of hypoglycaemia and risks unconsciousness. However, it is also specifically contemplated herein that, in some cases, the composition of the invention may be administered by a third party or for administration by a third party (medically qualified or otherwise), for example, when the hypoglycaemic subject is unable or would have difficulty self-administering the glucagon peptide-containing composition of the present invention.

In accordance with any one of the second to sixth aspects of the invention, the nanoparticle composition may be administered or for administration with (i.e. simultaneously, separately or sequentially) one or more therapeutic agents for the control of diabetes.

In accordance with any one of the second to sixth aspects of the invention, the nanoparticle composition may be administered or for administration by any suitable route. In particular cases, the nanoparticle composition may be administered or for administration via a route selected from the group consisting of: intravenous (i.v.), intramuscular (i.m.), intradermal (i.d.), intraperitoneal or subcutaneous (s.c.) injection or infusion; buccal; sublabial; sublingual; by inhalation; via one or more mucosal membranes; urogenital; rectal; intranasal and dermal.

In a seventh aspect, the present invention provides an article of manufacture comprising:
a nanoparticle composition as defined in accordance with the first aspect of the invention;
a container for housing the nanoparticle composition; and
an insert and/or label. Preferably, the insert and/or label provide instructions, dosage and/or administration information relating to the use of the nanoparticle composition in a method of treatment of hypoglycaemia. In particular, the acute treatment of a hypoglycaemic adverse event, such as diabetic hypoglycaemia.

In an eighth aspect, the present invention provides a process for producing a nanoparticle composition as defined in accordance with the first aspect of the invention, the process comprising:
providing a nanoparticle comprising a core comprising a metal and/or a semiconductor and a corona comprising a plurality of ligands covalently linked to the core, wherein said ligands comprise glutathione and, optionally, further comprise one or more carbohydrate-containing ligands (e.g. glucose-containing ligands); and
contacting the nanoparticle with at least one glucagon peptide under conditions which allow the at least one glucagon peptide to bind to the corona of the nanoparticle.

In some cases, in accordance with this aspect of the present invention, the process comprises an earlier step of producing the nanoparticle, said earlier step comprising: combining a solution comprising glutathione and, optionally, further comprising one or more derivatised carbohydrate moieties (e.g. thioethyl-glucose) with a solution comprising a core-forming material (e.g. gold III chloride) and with a reducing agent (e.g. sodium borohydride), thereby causing the nanoparticle to self-assemble.

The present invention includes the combination of the aspects and preferred features described except where such a combination is clearly impermissible or is stated to be expressly avoided. These and further aspects and embodiments of the invention are described in further detail below and with reference to the accompanying examples and figures.

### BRIEF DESCRIPTION OF THE FIGURES

FIG. 1 shows the glucagon binding curve with % glucagon bound (y-axis) plotted against Au added (x-axis) for gold nanoparticles having a mixed corona of 90% glutathione ligands and 10% glucose-C2 ligands.

FIG. 2 shows the glucagon binding capacity curve with number of glucagon peptides bound per nanoparticle (y-axis) plotted against the number of glucagon peptides added per nanoparticle (x-axis) for gold nanoparticles having a mixed corona of 90% glutathione ligands and 10% glucose-C2 ligands.

### DETAILED DESCRIPTION OF THE INVENTION

In describing the present invention, the following terms will be employed, and are intended to be defined as indicated below.

As used herein, “nanoparticle” refers to a particle having a nanomeric scale, and is not intended to convey any specific shape limitation. In particular, “nanoparticle” encompasses nanospheres, nanotubes, nanoboxes, nanoclusters, nanorods and the like. In certain embodiments the nanoparticles and/or nanoparticle cores contemplated herein have a generally polyhedral or spherical geometry.

Nanoparticles comprising a plurality of carbohydrate-containing ligands have been described in, for example, WO 2002/032404, WO 2004/108165, WO 2005/116226, WO 2006/037979, WO 2007/015105, WO 2007/122388, WO 2005/091704 (the entire contents of each of which is expressly incorporated herein by reference) and such nanoparticles may find use in accordance with the present invention. Moreover, gold-coated nanoparticles comprising a magnetic core of iron oxide ferrites (having the formula XFe₂O₄, where X=Fe, Mn or Co) functionalised with organic compounds (e.g. via a thiol-gold bond) are described in EP2305310 (the entire contents of which is expressly incorporated herein by reference) and are specifically contemplated for use as nanoparticles/nanoparticle cores in accordance with the present invention.

As used herein, “corona” refers to a layer or coating, which may partially or completely cover the exposed surface of the nanoparticle core. The corona includes a plurality of ligands which generally include at least one carbohydrate moiety, one surfactant moiety and/or one glutathione moiety. Thus, the corona may be considered to be an organic layer that surrounds or partially surrounds the metallic and/or semiconductor core. In certain embodiments the corona provides and/or participates in “passivating” the core of the nanoparticle. Thus, in certain cases the corona may include a sufficiently complete coating layer substantially to stabilise the semiconductor or metal-containing core. However, it is specifically contemplated herein that certain nanoparticles having cores, e.g., that include a metal oxide-containing inner core coated with a noble metal may include a corona that only partially coats the core surface. In certain cases the corona facilitates solubility, such as water solubility, of the nanoparticles of the present invention.

### Nanoparticles

Nanoparticles are small particles, e.g. clusters of metal or semiconductor atoms, that can be used as a substrate for immobilising ligands.

Preferably, the nanoparticles have cores having mean diameters between 0.5 and 50 nm, more preferably between 0.5 and 10 nm, more preferably between 0.5 and 5 nm, more preferably between 0.5 and 3 nm and still more preferably between 0.5 and 2.5 nm. When the ligands are considered in addition to the cores, preferably the overall mean diameter of the particles is between 2.0 and 20 nm, more preferably between 3 and 10 nm and most preferably between 4 and 5 nm. The mean diameter can be measured using techniques well known in the art such as transmission electron microscopy.

The core material can be a metal and/or semiconductor (said semiconductor optionally comprising metal atoms or being an organic semiconductor) and may be formed of more than one type of atom. Preferably, the core material is a metal selected from Au, Fe or Cu. Nanoparticle cores may also be formed from alloys including Au/Fe, Au/Cu, Au/Gd, Au/Fe/Cu, Au/Fe/Gd and Au/Fe/Cu/Gd, and may be used in the present invention. Preferred core materials are Au and Fe, with the most preferred material being Au. The cores of the nanoparticles preferably comprise between about 100 and 500 atoms (e.g. gold atoms) to provide core diameters in the nanometre range. Other particularly useful core materials are doped with one or more atoms that are NMR active, allowing the nanoparticles to be detected using NMR, both in vitro and in vivo. Examples of NMR active atoms include Mn⁺², Gd⁺³, Eu⁺², Cu⁺², V⁺², Co⁺², Ni⁺², Fe⁺², Fe⁺³ and lanthanides⁺³, or the quantum dots described elsewhere in this application.

Nanoparticle cores comprising semiconductor compounds can be detected as nanometre scale semiconductor crystals are capable of acting as quantum dots, that is they can absorb light thereby exciting electrons in the materials to higher energy levels, subsequently releasing photons of light at frequencies characteristic of the material. An example of a semiconductor core material is cadmium selenide, cadmium sulphide, cadmium tellurium. Also included are the zinc compounds such as zinc sulphide.

In some embodiments, the core of the nanoparticles may be magnetic and comprise magnetic metal atoms, optionally in combination with passive metal atoms. By way of example, the passive metal may be gold, platinum, silver or copper, and the magnetic metal may be iron or gadolinium. In preferred embodiments, the passive metal is gold and the magnetic metal is iron. In this case, conveniently the ratio of passive metal atoms to magnetic metal atoms in the core is between about 5:0.1 and about 2:5. More preferably, the ratio is between about 5:0.1 and about 5:1. As used herein, the term “passive metals” refers to metals which do not show magnetic properties and are chemically stable to oxidation. The passive metals may be diamagnetic or superparamagnetic. Preferably, such nanoparticles are superparamagnetic.

Examples of nanoparticles which have cores comprising a paramagnetic metal, include those comprising Mn⁺², Gd⁺³, Eu⁺², Cu⁺², V⁺², Co⁺², Ni⁺², Fe⁺², Fe⁺³ and lanthanides⁺³.

Other magnetic nanoparticles may be formed from materials such as MnFe (spinel ferrite) or CoFe (cobalt ferrite) can be formed into nanoparticles (magnetic fluid, with or without the addition of a further core material as defined above. Examples of the self-assembly attachment chemistry for producing such nanoparticles is given in Biotechnol. Prog., 19:1095-100 (2003), J. Am. Chem. Soc. 125:9828-33 (2003), J. Colloid Interface Sci. 255:293-8 (2002).

In some embodiments, the nanoparticle or its ligand comprises a detectable label. The label may be an element of the core of the nanoparticle or the ligand. The label may be detectable because of an intrinsic property of that element of the nanoparticle or by being linked, conjugated or associated with a further moiety that is detectable. Preferred examples of labels include a label which is a fluorescent group, a radionuclide, a magnetic label or a dye. Fluorescent groups include fluorescein, rhodamine or tetramethyl rhodamine, Texas-Red, Cy3, Cy5, etc., and may be detected by excitation of the fluorescent label and detection of the emitted light using Raman scattering spectroscopy (Y. C. Cao, R. Jin, C. A. Mirkin, Science 2002, 297: 1536-1539).

In some embodiments, the nanoparticles may comprise a radionuclide for use in detecting the nanoparticle using the radioactivity emitted by the radionuclide, e.g. by using PET, SPECT, or for therapy, i.e. for killing target cells. Examples of radionuclides commonly used in the art that could be readily adapted for use in the present invention include ^{99m}Tc, which exists in a variety of oxidation states although the most stable is TcO⁴⁻; ³²P or ³³P; ⁵⁷Co; ⁵⁹Fe; ⁶⁷Cu which is often used as Cu²⁺ salts; ⁶⁷Ga which is commonly used a Ga³⁺ salt, e.g. gallium citrate; ⁶⁸Ge; ⁸²Sr; ⁹⁹Mo; ¹⁰³Pd; ¹¹¹In which is generally used as In³⁺ salts; ¹²⁵I or ¹³¹I which is generally used as sodium iodide; ¹³⁷Cs; ¹⁵³Gd; ¹⁵³Sm; ¹⁵⁸Au; ¹⁸⁶Re; ²⁰¹Tl generally used as a Tl⁺ salt such as thallium chloride; ³⁹Y³⁺; ⁷¹Lu³⁺; and ²⁴Cr²⁺. The general use of radionuclides as labels and tracers is well known in the art and could readily be adapted by the skilled person for use in the aspects of the present invention. The radionuclides may be employed most easily by doping the cores of the nanoparticles or including them as labels present as part of ligands immobilised on the nanoparticles.

Additionally or alternatively, the nanoparticles of the present invention, or the results of their interactions with other species, can be detected using a number of techniques well known in the art using a label associated with the nanoparticle as indicated above or by employing a property of them. These methods of detecting nanoparticles can range from detecting the aggregation that results when the nanoparticles bind to another species, e.g. by simple visual inspection or by using light scattering (transmittance of a solution containing the nanoparticles), to using sophisticated techniques such as transmission electron microscopy (TEM) or atomic force microscopy (AFM) to visualise the nanoparticles. A further method of detecting metal particles is to employ plasmon resonance that is the excitation of electrons at the surface of a metal, usually caused by optical radiation. The phenomenon of surface plasmon resonance (SPR) exists at the interface of a metal (such as Ag or Au) and a dielectric material such as air or water. As changes in SPR occur as analytes bind to the ligand immobilised on the surface of a nanoparticle changing the refractive index of the interface. A further advantage of SPR is that it can be used to monitor real time interactions. As mentioned above, if the nanoparticles include or are doped with atoms which are NMR active, then this technique can be used to detect the particles, both in vitro or in vivo, using techniques well known in the art. Nanoparticles can also be detected using a system based on quantitative signal amplification using the nanoparticle-promoted reduction of silver (I). Fluorescence spectroscopy can be used if the nanoparticles include ligands as fluorescent probes. Also, isotopic labelling of the carbohydrate can be used to facilitate their detection.

### Glucagon Peptide

In certain cases in accordance with the present invention, the “glucagon peptide” may be selected from the group consisting of:
(i) a peptide comprising or consisting of an amino acid sequence having at least 70%, 80%, 90%, 95% or 99% amino acid sequence identity to the full-length sequence set forth in SEQ ID NO: 1;
(ii) a peptide comprising or consisting of the full-length amino acid sequence set forth in SEQ ID NO: 1;
(iii) a peptide comprising or consisting of a variant sequence of the full-length amino acid sequence set forth in SEQ ID NO: 1, wherein said variant differs by addition, deletion, substitution or modification (e.g. chemical modification or derivatisation) of not more than 1, 2, 3, 4, 5, 6, 7, 8, 9 or not more than 10 amino acids from said full-length amino acid sequence set forth in SEQ ID NO: 1;
(iv) a peptide comprising or consisting of a fragment of any one of (i)-(iii), said fragment having a sequence length of at least 15, 20, 25 or 28 amino acids.

Sequence identity may be calculated using any suitable method, as would be readily apparent to the skilled person. In certain cases, amino acid sequence identity between a candidate sequence and a reference sequence, e.g. the sequence of SEQ ID NO: 1, may be calculated using the online tool SUPERMATCHER available at the following URL: http://emboss.bioinformatics.nl/cgi-bin/emboss/supermatcher using GAP opening penalty of 10.0 and GAP extension penalty of 0.5 (see EMBOSS: The European Molecular Biology Open Software Suite (2000) Rice, P. Longden, I. and Bleasby, A. Trends in Genetics 16, (6) pp. 276-277).

Preferably, said glucagon peptide of any one of (i)-(iv) exhibits biological activity of glucagon. In particular, said glucagon peptide of any one of (i)-(iv) may exhibit at least 50% of the activity of the glucagon peptide of SEQ ID NO: 1 in an in vitro or in vivo bioassay of glucagon activity. In certain cases, the glucagon activity may comprise one or more activities selected from the group consisting of: glucagon receptor agonist activity (e.g. activation of the human glucagon receptor having the amino acid sequence set forth at UniProt accession no. P47871, version 1, 1 Feb. 1996); stimulation of gluconeogenesis; stimulation of glycogenolysis; elevation of blood glucose concentration; and suppression of liver glycolysis. In particular, the glucagon peptide activity may comprise the ability to elevate blood glucose concentration and/or restore normal blood glucose concentration in a hypoglycaemic mammal, e.g., a diabetic human experiencing a hypoglycaemic episode.

The glucagon peptide is bound to the corona of the nanoparticle. Without wishing to be bound by any theory, it is presently believed that the glucagon peptide may participate in one or more reversible binding interactions with one or more ligands that provide the corona of the nanoparticle. In particular, a portion of the sequence of amino acids may participate in hydrogen bonding, Van der Waals forces and/or electrostatic interactions with one or more ligands (e.g. interacting with one or more glutathione ligands and/or carbohydrate-containing ligands, e.g. glucose moeities). The peptide binding may involve adsorption, absorption or other direct or indirect interaction with one or more ligands of the nanoparticle.

As described herein with reference to certain embodiments of the present invention, the glucagon peptide may be bound such that at least a fraction or portion of the bound glucagon peptide is released from the nanoparticle upon contacting the nanoparticle with a physiological solution, e.g. when administered to a subject to be treated. As described herein the glucagon peptide may be bound to the nanoparticle in a manner such that the glucagon peptide is stabilised (e.g. thermostabilised and/or stabilised against the formation of glucagon fibrils) while bound, but is releasable and available in a form that is biologically active (for example, releasable such that the glucagon peptide is detectable by ELISA and/or capable of exerting at least one biological action in an in vitro or in vivo assay system that is characteristic of the free glucagon peptide). In particular, the glucagon peptide may be bound to the nanoparticle such that a suspension of the glucagon-bound nanoparticles gives a positive result in an ELISA for, e.g., (human) glucagon and/or exerts an effect on a glucagon receptor (e.g. expressed on a cell surface) and/or exerts an elevating effect on blood glucose concentration in a mammalian subject.

### Administration and Treatment

The nanoparticles and compositions of the invention may be administered to patients by any number of different routes, including enteral or parenteral routes. Parenteral administration includes administration by the following routes: intravenous, cutaneous or subcutaneous, nasal, intramuscular, intraocular, transepithelial, intraperitoneal and topical (including dermal, ocular, rectal, nasal, inhalation and aerosol), film, patch and rectal systemic routes. In some cases the nanoparticles and compositions of the invention may be administered or for administration via transbuccal route.

Administration be performed e.g. by injection, or ballistically using a delivery gun to accelerate their transdermal passage through the outer layer of the epidermis. The nanoparticles may also be delivered in aerosols. This is made possible by the small size of the nanoparticles.

The nanoparticles of the invention may be formulated as pharmaceutical compositions that may be in the forms of solid or liquid compositions. In some cases the nanoparticles may be formulated in a viscoelastic film, e.g. for transbuccal delivery. Compositions in accordance with the present invention will generally comprise a carrier of some sort, for example a solid carrier or a liquid carrier such as water, petroleum, animal or vegetable oils, mineral oil or synthetic oil. Physiological saline solution, or glycols such as ethylene glycol, propylene glycol or polyethylene glycol may be included. Such compositions and preparations generally contain at least 0.1 wt % of the compound.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, solutions of the compounds or a derivative thereof, e.g. in physiological saline, a dispersion prepared with glycerol, liquid polyethylene glycol or oils.

In addition to one or more of the compounds, optionally in combination with other active ingredient, the compositions can comprise one or more of a pharmaceutically acceptable excipient, carrier, buffer, stabiliser, isotonicising agent, preservative or anti-oxidant or other materials well known to those skilled in the art. Such materials should be non-toxic and should not interfere with the efficacy of the active ingredient. The precise nature of the carrier or other material may depend on the route of administration, e.g. intravenously, orally or parenterally.

Liquid pharmaceutical compositions are typically formulated to have a pH between about 3.0 and 9.0, more preferably between about 4.5 and 8.5 and still more preferably between about 5.0 and 8.0. The pH of a composition can be maintained by the use of a buffer such as acetate, citrate, phosphate, succinate, Tris or histidine, typically employed in the range from about 1 mM to 50 mM. The pH of compositions can otherwise be adjusted by using physiologically acceptable acids or bases.

Preservatives are generally included in pharmaceutical compositions to retard microbial growth, extending the shelf life of the compositions and allowing multiple use packaging. Examples of preservatives include phenol, meta-cresol, benzyl alcohol, para-hydroxybenzoic acid and its esters, methyl paraben, propyl paraben, benzalconium chloride and benzethonium chloride. Preservatives are typically employed in the range of about 0.1 to 1.0% (w/v).

Preferably, the pharmaceutically compositions are given to an individual in a prophylactically effective amount or a therapeutically effective amount (as the case may be, although prophylaxis may be considered therapy), this being sufficient to show benefit to the individual. Typically, this will be to cause a therapeutically useful activity providing benefit to the individual. The actual amount of the compounds administered, and rate and time-course of administration, will depend on the nature and severity of the condition being treated. Prescription of treatment, e.g. decisions on dosage etc., is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Handbook of Pharmaceutical Additives, 2nd Edition (eds. M. Ash and I. Ash), 2001 (Synapse Information Resources, Inc., Endicott, N.Y., USA); Remington's Pharmaceutical Sciences, 20th Edition, 2000, pub. Lippincott, Williams & Wilkins; and Handbook of Pharmaceutical Excipients, 2nd edition, 1994. By way of example, and the compositions are preferably administered to patients in dosages of between about 0.01 and 100 mg of active compound per kg of body weight, and more preferably between about 0.5 and 10 mg/kg of body weight.

The following is presented by way of example and is not to be construed as a limitation to the scope of the claims.

### EXAMPLES

### Example 1

### Synthesis of Nanoparticles

Synthesis of gold nanoparticles having a corona of carbohydrate ligands and/or glutathione ligands has been described previously (WO 2011/154711; and Lund et al., 2011, Biomaterials Vol. 32 pp. 9776-9784, the entire contents of which are expressly incorporated herein by reference).

The isoelectric point (ₚI) of glucagon lies close to neutral pH (approximately 7±1). This presents a challenge for the design of an optimal nanoparticle corona composition. For example, the alpha-galactose/EG6NH₂ nanoparticles described in WO2011/154711 would be considered sub-optimal for binding glucagon. Therefore, the present inventors sought an alternative nanoparticle corona composition adapted to bind glucagon. To this end, nanoparticles having a corona comprising 90% glutathione and 10% glucoseC2 ligands were prepared as follows.

Oxidised GSH was initially dissolved in 1.4 ml of H₂O, and subsequently mixed with 14.3 ml of Methanol (52.5 mg, 15.7 ml, 5.45 mM). To this solution 1.6 ml of GlucoseC2 (i.e. 2′-thioethyl-α-D-glucopyranoside—see WO2011/154711 for structure) in methanol (4.6 mg, 1.6 ml, 12 mM) was added, and stirred for 2 minutes.

After allowing the solution to mix for 5 minutes, 1.15 ml of aqueous HAuCl₄ (21.6 mg, 1.15 ml, 55 mM) was added under stirring, and mixed for a further 5 minutes.

The reduction of the reaction mixture was then performed by the addition of 1.4 ml aqueous NaBH₄ (52.8 mg, 1.4 ml, 1M) in one rapid addition under vigorous stirring.

The reaction mixture was then set to stir for an hour on a rotary shaker before centrifuging and removing the supernatant from the precipitated GSH/glucoseC2 nanoparticles (NPs).

The collected NP pellet was then washed with 90% methanol, and subsequently redissolved in H₂O. The GSH/glucoseC2 NPs were then filtered through a 10 kDa Millipore amicon centrifuge filter and washed 5 times with H₂O before finally collecting the material in 3 ml H₂O.

### Example 2

### Glucagon Peptide Binding to Nanoparticles

The present inventors have investigated the ability of the peptide glucagon to bind nanoparticles.

Glucagon has the following sequence:

| | |
|---|---|
| HSQGTFTSDYSKYLDSRRAQDFVQWLMNT. | (SEQ ID NO: 1) |

### Glucagon Binding

The standard insulin binding assay, essentially as described in examples 3 and 4 of WO2011/154711, was performed with the crucial modifications of using a 2 mg/ml solution of Glucagon in a 50 mM NaAc buffer solution (pH 4.6), instead of the standard insulin solution, and the use of the newly produced GSH/GlucoseC2 NPs instead of the αGal/AL GNPs.

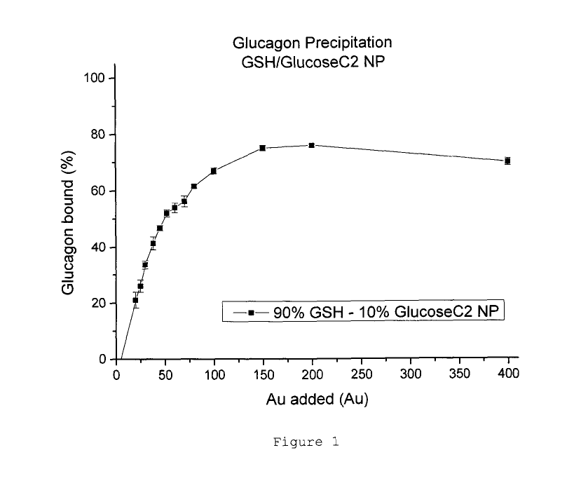

Glucagon precipitation was achieved successfully using the adapted method. The results indicate a plateau at ˜80% of total Glucagon under these conditions (see FIG. 1).

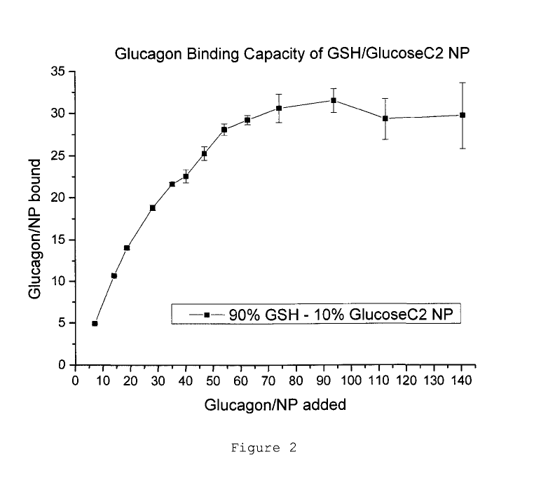

Converting the data to a binding capacity curve gives a better indication of how well the GSH/GlucoseC2 NPs bind Glucagon. Based on the data, a binding capacity of 30 Glucagon per NP was established (see FIG. 2).

All references cited herein are incorporated herein by reference in their entirety and for all purposes to the same extent as if each individual publication or patent or patent application was specifically and individually indicated to be incorporated by reference in its entirety.

The specific embodiments described herein are offered by way of example, not by way of limitation. Any sub-titles herein are included for convenience only, and are not to be construed as limiting the disclosure in any way.

1. A nanoparticle composition comprising:
(a) a nanoparticle comprising:
(i) a core comprising a metal and/or a semiconductor;
(ii) a corona comprising a plurality of ligands covalently linked to the core, wherein said plurality of ligands comprise at least one glutathione; and
(b) at least one glucagon peptide that is non-covalently bound to the corona.

2. The nanoparticle composition according to claim 1, wherein said plurality of ligands further comprises at least one carbohydrate-containing ligand.

3. The nanoparticle composition according to claim 2, wherein said carbohydrate-containing ligand comprises a monosaccharide covalently linked to said core via a thioalkyl linker.

4. The nanoparticle composition according to claim 3, wherein said carbohydrate-containing ligand comprises 2′-thioethyl-α-D-glucopyranoside (glucoseC2).

5. The nanoparticle composition according to claim 2, wherein said glutathione ligand and said carbohydrate-containing ligand are present at a ratio of between 100:1 and 1:1.

6. The nanoparticle composition according to claim 5, wherein said corona is formed of 75%-95% glutathione ligands and 25%-5% carbohydrate-containing ligands.

7. The nanoparticle composition according to claim 6, wherein said corona is formed of 90% glutathione ligands and 10% 2′-thioethyl-α-D-glucopyranoside ligands.

8. The nanoparticle composition according to claim 1, wherein the glucagon peptide comprises or consists of:
(i) an amino acid sequence having at least 70%, 80%, 90%, 95% or 99% amino acid sequence identity to the full-length sequence HSQGTFTSDYSKYLDSRRAQDFVQWLMNT (SEQ ID NO: 1);
(ii) a peptide comprising or consisting of the full-length amino acid sequence set forth in SEQ ID NO: 1;
(iii) a peptide comprising or consisting of a variant sequence of the full-length amino acid sequence set forth in SEQ ID NO: 1, wherein said variant differs by addition, deletion, substitution or modification of not more than 1, 2, 3, 4, 5, 6, 7, 8, 9 or not more than 10 amino acids from said full-length amino acid sequence set forth in SEQ ID NO: 1;
(iv) a peptide comprising or consisting of a fragment of any one of (i)-(iii), said fragment having a sequence length of at least 15, 20, 25 or 28 amino acids.

9.-12. (canceled)

13. The nanoparticle composition according to claim 1, wherein the number of glucagon peptide molecules bound to the nanoparticle is selected from: 1, 2, 3, 4, 5, 10, 15, 20, 25 or at least 30 per core.

14.-25. (canceled)

26. The nanoparticle composition according to claim 1, wherein the nanoparticle composition comprises a carrier in which the nanoparticles and bound glucagon peptides are suspended or embedded.

27. (canceled)

28. The nanoparticle composition according to claim 1, wherein the composition is in the form of one or more doses of a defined quantity of glucagon peptide or of a defined level of glucagon peptide activity units.

29. The nanoparticle composition according to claim 1, wherein the composition further comprises at least one permeation enhancer that is non-covalently or covalently bound to said core and/or or said corona.

30.-34. (canceled)

35. A method of treatment of hypoglycaemia in a mammalian subject,
the method comprising administering a therapeutically effective amount of a nanoparticle composition as defined in claim 1 to the subject in need of said treatment.

36. A method of increasing blood glucose concentration in a mammalian subject, the method comprising administering an effective amount of a nanoparticle composition as defined in claim 1 to the subject.

37. (canceled)

38. The method in accordance with claim 35, wherein the subject is diabetic.

39. The method in accordance with claim 35, wherein said hypoglycaemia is a hypoglycaemic adverse event.

40. The method in accordance with claim 35, wherein the nanoparticle composition is administered via a route selected from the group consisting of: intravenous (i.v.), intramuscular (i.m.), intradermal (i.d.), intraperitoneal or subcutaneous (s.c.) injection or infusion; buccal; sublabial; sublingual; by inhalation; via one or more mucosal membranes; urogenital; rectal; intranasal; and dermal.

41. The method in accordance with claim 35, wherein the nanoparticle composition is provided in a “ready to use” form and is administered without a preceding reconstitution step.

42. An article of manufacture comprising:
a nanoparticle composition as defined in claim 1;
a container for housing the nanoparticle composition; and
an insert and/or label.

43. The article of manufacture according to claim 42, wherein the insert and/or label provides instructions, dosage and/or administration information relating to the use of the nanoparticle composition in a method of treatment of hypoglycaemia.

44. A process for producing a nanoparticle composition as defined in claim 1, the process comprising:
providing a nanoparticle comprising a core comprising a metal and/or a semiconductor and a corona comprising a plurality of ligands covalently linked to the core, wherein said plurality of ligands comprise at least one glutathione; and
contacting the nanoparticle with at least one glucagon peptide under conditions which allow the at least one glucagon peptide to bind to the corona of the nanoparticle.

45.-46. (canceled)
